SEQUENCE LISTING (10 sequences)

SEQ ID NO: 1 - protein, 519 aa, Homo Sapiens
MGLEMSSKDSPGSLDGRAWEDAQKPQSAWCGGRKTRVYATSSRRAPPSEGTRRGGAARPEEAAEEGPPAAPGSLRHSGPLGPHACPTALPEPQVTSAMSSQVVGIEPLYIKAEPASPDSPKGSSETETEPPVALAPGPAPTRCLPGHKEEEDGEGAGPGEQGGGKLVLSSLPKRLCLVCGDVASGYHYGVASCEACKAFFKRTIQGSIEYSCPASNECEITKRRRKACQACRFTKCLRVGMLKEGVRLDRVRGGRQKYKRRPEVDPLPFPGPFPAGPLAVAGGPRKTAPVNALVSHLLVVEPEKLYAMPDPAGPDGHLPAVATLCDLFDREIVVTISWAKSIPGFSSLSLSDQMSVLQSVWMEVLVLGVAQRSLPLQDELAFAEDLVLDEEGARAAGLGELGAALLQLVRRLQALRLEREEYVLLKALALANSDSVHIEDAEAVEQLREALHEALLEYEAGRAGPGGGAERRRAGRLLLTLPLLRQTAGKVLAHFYGVKLEGKVPMHKLFLEMLEAMMD

SEQ ID NO: 2 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 3 - DNA, 49 bp, Artificial Sequence
  Oligonucleotide primer
ggggacaagtttgtacaaaaaagcaggctatgggattggagatgagctc

SEQ ID NO: 4 - DNA, 50 bp, Artificial Sequence
  Oligonucleotide primer
ggggaccactttgtacaagaaagctgggttcagtccatcatggcctcgag

SEQ ID NO: 5 - DNA, 30 bp, Artificial Sequence
  Oligonucleotide primer
acgggtagaggtcactgtgacctctacccg

SEQ ID NO: 6 - DNA, 30 bp, Artificial Sequence
  Oligonucleotide primer
cgggtagaggtcacagtgacctctacccgt

SEQ ID NO: 7 - DNA, 2402 bp, Homo sapiens
agctcacagcaagtccaggctagaggtagaaacgtgagagccccacggctggggaagattgccatgggattggagatgagctccaaggacagccctggcagtctggatggaagagcttgggaagatgctcagaaaccacaaagtgcctggtgcggtgggaggaaaaccagagtgtatgctacaagcagccggcgggcgccgccgagtgaggggacgcggcgcggtggggcggcgcggcccgaggaggcggcggaggaggggccgcccgcggcccccggctcactccggcactccgggccgctcggcccccatgcctgcccgaccgcgctgccggagccccaggtgaccagcgccatgtccagccaggtggtgggcattgagcctctctacatcaaggcagagccggccagccctgacagtccaaagggttcctcggagacagagaccgagcctcctgtggccctggcccctggtccagctcccactcgctgcctcccaggccacaaggaagaggaggatggggagggggctgggcctggcgagcagggcggtgggaagctggtgctcagctccctgcccaagcgcctctgcctggtctgtggggacgtggcctccggctaccactatggtgtggcatcctgtgaggcctgcaaagccttcttcaagaggaccatccaggggagcatcgagtacagctgtccggcctccaacgagtgtgagatcaccaagcggagacgcaaggcctgccaggcctgccgcttcaccaagtgcctgcgggtgggcatgctcaaggagggagtgcgcctggaccgcgtccggggtgggcggcagaagtacaagcggcggccggaggtggacccactgcccttcccgggccccttccctgctgggcccctggcagtcgctggaggcccccggaagacagcagccccagtgaatgcactggtgtctcatctgctggtggttgagcctgagaagctctatgccatgcctgaccccgcaggccctgatgggcacctcccagccgtggctaccctctgtgacctctttgaccgagagattgtggtcaccatcagctgggccaagagcatcccaggcttctcatcgctgtcgctgtctgaccagatgtcagtactgcagagcgtgtggatggaggtgctggtgctgggtgtggcccagcgctcactgccactgcaggatgagctggccttcgctgaggacttagtcctggatgaagagggggcacgggcagctggcctgggggaactgggggctgccctgctgcaactagtgcggcggctgcaggccctgcggctggagcgagaggagtatgttctactaaaggccttggcccttgccaattcagactctgtgcacatcgaagatgagccgaggctgtggagcagctgcgagaagctcctgcacgaggccctgctggagtatgaagccggccgggctggccccggagggggtgctgagcggcggcgggcgggcaggctgctgctcacgctaccgctcctccgccagacagcgggcaaagtgctggcccatttctatggggtgaagctggagggcaaggtgcccatgcacaagctgttcttggagatgctcgaggccatgatggactgaggcaaggggtgggactggtgggggttctggcaggacctgcctagcatggggtcagccccaagggctggggcggagctggggtctgggcagtgcacagcctgctggcagggccagggctaatgccatcagcccctgggaacaggccccacgccctctcctccccctcctagggggtgtcagaagctgggaacgtgtgtccaggctctgggcacagtgctgccccttgcaagccataacggtgcccccagagtgtagggggccttgcggaagccatagggggctgcacgggatgcgtgggaggcagaaacctatctcagggagggaaggggatggaggccagagtctcccagtgggtgatgcttttgctgctgcttaatcctaccccctcttcaaagcagagtgggacttggagagcaaaggcccatgcccccttcgctcctcctctcatcatttgcattgggcattagtgtccccccttgaagcaataactccaagcagactccagcccctggacccctggggtggccagggcttccccatcagctcccaacgagcctcctcagggggtaggagagcactgcctctatgccctgcagagcaataacactatatttatttttgggtttggccagggaggcgcagggacatggggcaagccagggcccagagcccttggctgtacagagactctattttaatgtatatttgctgcaaagagaaaccgcttttggttttaaacctttaatgagaaaaaaatatataataccgagctc

SEQ ID NO: 8 - DNA, 2481 bp, Homo sapiens
cgaacttggggggagtgcacagaagaacttcgggagcgcacgcgggaccagggaccaggctgagactcggggcgccagtccgggcaggggcagcgggagccggccggagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcttccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgcctgggaacacgtgtgcacatgcgcactctctcatatgccaccccatgtgcctttagtccacggaccccagagcacccccaagcctgggcttagctgcagaacagagggacctgctcacctgcccaaaggggatgaagggagggaggctcaaggcccttgggggagggggatgccttcatgggggtgacccacgatgtgttcttatcccccccgcctggccaccggcctttatgttttttgtaagataaaccgtttttaacacatagcgccgtgctgtaaataagcccagtactgctgtaaatacaggaagaaagagcttgaggtgggagcgggctgggaggaagggatgggccccggccttcctgggcagcctttccagcctcctgctgggctctctcttcctaccctccttccacatgtacatgtacataaactgtcactctaggaagaagacaaatgacagattctgaccatttatatttgtgtattttccaggatttatagtatgtgacttttctgattaatatatttaatatattgaataaaaaatagacatgtagttgg

SEQ ID NO: 9 - DNA, 63 bp, Artificial Sequence
  Oligonucleotide primer
ggccagtgaattgtaatacgactcactatagggaggcggtttttttttttttttttttttttt

SEQ ID NO: 10 - DNA, 25 bp, Artificial Sequence
  Oligonucleotide primer
gtcgtcaagatgctaccgttcagga

(19) United States
(12) Patent Application Publication
(10) Pub. No.: US 2003/0103965 A1
(43) Pub. Date: Jun. 5, 2003

(54) Method for identifying substances which positively influence inflammatory conditions

(75) Inventors: Birgit Jung, Schwabenheim (DE); Norbert Kraut, Vienna (AT); Stefan Mueller, Munich (DE)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Boehringer Ingelheim International GmbH

(21) Appl. No.: 10/211,239

(22) Filed: Aug. 5, 2002

Related U.S. Application Data
(60) Provisional application No. 60/315,775, filed on Aug. 30, 2001.

(30) Foreign Application Priority Data
Aug. 6, 2001 (EP) 01 119 003.0

(51) Int. Cl. A61K 39/395; A61K 31/00; G01N 33/53; G01N 33/567

(52) U.S. Cl. 424/130.1; 435/7.2; 514/1

(57) ABSTRACT

The present invention relates to NHR-proteins involved in inflammatory processes and the modulation of the function of such NHR-protein in order to positively influence inflammatory diseases.

### BACKGROUND OF THE INVENTION

1. Field of the Invention

The present invention belongs to the field of modulation of inflammatory processes, in particular of chronic inflammatory airway diseases, in which macrophages play an important role. The inflammatory processes can be modulated according to the invention by influencing the biological activity of a nuclear hormone receptor protein, which is identified to be involved in the inflammatory process.

1. Background

Examples for chronic inflammatory airway diseases, in which macrophages play an important role is chronic bronchitis (CB). CB may occur with or without airflow limitation and includes chronic obstructive pulmonary disease (COPD). CB is a complex disease encompassing symptoms of several disorders: chronic bronchitis which is characterized by cough and mucus hypersecretion, small airway disease, including inflammation and peribronchial fibrosis, emphysema, and airflow limitation. CB is characterized by an accelerated and irreversible decline of lung function. The major risk factor for developing CB is continuous cigarette smoking. Since only about 20% of all smokers are inflicted with CB, a genetic predisposition is also likely to contribute to the disease.

The initial events in the early onset of CB are inflammatory, affecting small and large airways. An irritation caused by cigarette smoking attracts macrophages and neutrophils, the number of which is increased in the sputum of smokers. Perpetual smoking leads to an ongoing inflammatory response in the lung by releasing mediators from macrophages, neutrophils and epithelial cells that recruit inflammatory cells to sites of the injury. So far there is no therapy available to reverse the course of CB. Smoking cessation may reduce the decline of lung function.

Only a few drugs are known to date to provide some relief for patients. Long-lasting β2-agonists and anticholinergics are applied to achieve a transient bronchodilation. A variety of antagonists for inflammatory events are under investigation like, LTB₄-inhibitors.

There is a continuous need to provide drugs for treating chronic inflammatory airway diseases. Chronic inflammatory airway diseases can be attributed to activated inflammatory immune cells, e.g. macrophages. There is, therefore, a need for drugs that modulate the function of macrophages in order to eliminate a source of inflammatory processes.

### SUMMARY OF THE INVENTION

In a first embodiment, the present invention relates to a method for determining whether a substance is an activator or an inhibitor of a function of a NHR-protein deregulated in a hyperactivated macrophage, characterized in that the method comprises contacting the NHR-protein or variant, mutant or fragment thereof having a NHR-protein function with a substance to be tested, whether it is an inhibitor or activator of a desired function of the NHR-protein, and measuring whether the desired function is inhibited or activated. In a specific embodiment, the inhibition or the activation of the desired function is measured directly. In another specific embodiment, the inhibition or the activation of the desired function is measured indirectly. In a preferred embodiment, the NHR-protein is a mammalian NHR-protein. In a specific embodiment of the invention, the NHR-protein is a human NHR-protein. In a preferred embodiment, the NHR-protein is selected from the group consisting of ERRα (SEQ ID NO:1); and NR4A1 (SEQ ID NO:2). In a specific embodiment the NHR-protein is ERRa (SEQ ID NO:1), a variant, a mutant or a fragment thereof having the same function. In another specific embodiment, the function is DNA recognition and/or DNA binding. In yet another specific embodiment, the function is protein recognition and/or protein binding. In another specific embodiment, the analysis is performed using a cellular system. In yet another specific embodiment, the analysis is performed using a cell-free system.

In a preferred embodiment of the invention, the present invention relates to a method for determining an expression level of a NHR-protein deregulated in a hyperactivated macrophage comprising determining the expression level of NHR-protein expressed in a macrophage. In a specific embodiment the macrophage is a mammalian macrophage. In a further embodiment the macrophage is a human macrophage. In yet another embodiment, the NHR-protein is selected from the group consisting of ERRα (SEQ ID NO:1); and NR4A1 (SEQ ID NO:2). In another embodiment, the expression level is determined by determining the level of nucleic acid coding for a NHR-protein in a macrophage. In yet another embodiment, the expression level is determined by determining the level of a NHR-protein. In a further embodiment the expression level is determined for diagnosis or monitoring of a chronic inflammatory airway disease. In a specific embodiment the chronic inflammatory airway disease is selected from the group consisting of chronic bronchitis and COPD. In yet another embodiment the analysis is performed using a macrophage or a part thereof obtainable from the site of inflammation.

In another preferred embodiment, the present invention relates to a test system for determining whether a substance is an activator or an inhibitor of a function of a NHR-protein deregulated in a hyperactivated macrophage comprising at least a NHR-protein or a variant, or a mutant, or a fragment thereof having a NHR-protein function. In a specific embodiment, the NHR-protein is selected from the group consisting of ERRα (SEQ ID NO:1); and NR4A1 (SEQ ID NO:2). In another specific embodiment, the test system comprises a cell expressing a NHR-protein.

In another preferred embodiment, the present invention relates to a substance determined to be an activator or inhibitor of a NHR-protein deregulated in a hyperactivated macrophage. In an equally preferred embodiment, the present invention relates to a substance which is an activator or inhibitor of a NHR-protein deregulated in a hyperactivated macrophage for the treatment for a disease. In a specific embodiment, the disease is a chronic inflammatory airway disease. In a more specific embodiment, the chronic inflammatory airway disease is selected from the group consisting of chronic bronchitis and COPD.

In another embodiment, the present invention relates to a pharmaceutical composition comprising at least one substance determined to be an activator or an inhibitor of a NHR-protein deregulated in a hyperactivated macrophage. In another embodiment, the present invention relates to the use of a substance determined to be an activator or inhibitor of a NHR-protein for preparing a pharmaceutical composition for treating a chronic inflammatory airway disease. In a specific embodiment, the chronic inflammatory airway disease is selected from the group consisting of chronic bronchitis and COPD.

In a preferred embodiment of the invention, the present invention relates to a method for treating a chronic inflammatory airway disease which method comprises administering to a being in need of such treatment a suitable amount of a pharmaceutical composition comprising at least one substance determined to be an activator or inhibitor of a NHR-protein. In a specific embodiment, the method is for treating a mammal. In a further embodiment, the method is for treating a human being. In another embodiment, the chronic inflammatory airway disease is selected from the group consisting of chronic bronchitis and COPD.

In an equally preferred embodiment, the present invention relates to a method for selectively modulating a NHR-protein in a macrophage, comprising administering a substance determined to be an activator or inhibitor of a NHR-protein. In a specific embodiment, the macrophage is involved in a chronic inflammatory airway disease. In another embodiment, the chronic inflammatory airway disease is selected from the group consisting of chronic bronchitis and COPD.

### DETAILED DESCRIPTION OF THE INVENTION

In the present invention it was found that macrophages involved in an inflammatory process, particularly in a chronic inflammatory airway disease, more particularly in chronic bronchitis or COPD, show a pattern of differentially expressed nucleic acid sequence and protein expression which differs from the pattern of gene expression of macrophages from healthy donors or donors in an irritated state, which later do contain macrophages in an activated state. Therefore, macrophages show different levels of activation under different inflammatory conditions. For example, it is shown in the present invention that macrophages involved in an inflammatory process in COPD smokers show different gene expression pattern than macrophages from healthy smokers, indicating that in COPD smokers macrophages are in a different, hereinafter named “hyperactivated” state. The present invention provides for the possibility to inhibit the hyperactivation or to reduce the hyperactive state of a macrophage by allowing the identification of substances which modulate a nuclear hormone receptor (NHR) protein involved in the hyperactivation or in maintaining the hyperactive state.

The term “chronic inflammatory airway disease” as used hereinafter includes, for example, Chronic Bronchitis (CB) and Chronic Obstructive Pulmonary Disease (COPD). The preferred meaning of the term “chronic inflammatory airway disease” is CB and COPD, the more preferred meaning is CB or COPD.

The invention is based on the identification of a nucleic acid sequence differentially expressed in a hyperactivated macrophage compared to a macrophage which is not hyperactivated. Such a nucleic acid sequence encodes for a nuclear receptor protein, which is involved in the hyperactivation or maintaining the hyperactive state of a macrophage involved in an inflammatory process, preferably in a chronic inflammatory airway disease. Such differentially expressed nucleic acid sequence or protein encoded by such nucleic acid sequence is in the following also named differentially expressed nucleic acid sequence or protein of the invention, respectively. In particular, the present invention teaches a link between phenotypic changes in macrophages due to differentially expressed nucleic acid sequence and protein expression pattern and involvement of macrophages in inflammatory processes and, thus, provides a basis for a variety of applications. For example, the present invention provides a method and a test system for determining the expression level of a macrophage protein of the invention or differentially expressed nucleic acid sequence of the invention and thereby provides e.g. for methods of diagnosis or monitoring of inflammatory processes with involvement of hyperactivated macrophages in mammalian, preferably human beings, especially such beings suffering from an inflammatory process, preferably in a chronic inflammatory airway disease, more preferably in chronic bronchitis or COPD. The invention also relates to a method for identifying a substance by means of a differentially expressed nucleic acid sequence or protein of the invention, which substance modulates, i.e. acts as an inhibitor or activator on the said differentially expressed nucleic acid sequence or protein of the invention and, thereby, positively influences chronic inflammatory processes by inhibition of the hyperactivation or reduction of the hyperactive state of macrophages and, thereby, allows treatment of mammals, preferably human beings, suffering from said disease. The invention also relates to a method for selectively modulating such a differentially expressed nucleic acid sequence or protein of the invention in a macrophage comprising administering a substance determined to be a modulator of said protein or differentially expressed nucleic acid sequence. The present invention includes the use of said substances for treating beings in need of a treatment for an inflammatory process.

In the present invention in a first step a differentially expressed nucleic acid sequence of the invention is identified which has a different expression pattern in a hyperactivated macrophage compared to a macrophage which is not hyperactivated. For the sake of conciseness this description deals particularly with investigation of macrophages involved in COPD, however, equivalent results may be obtained with samples from subjects suffering from other chronic inflammatory airway diseases, e.g. other chronic bronchitis symptoms. The investigation of the different expression pattern leads to the identification of a series of differentially expressed nucleic acid sequences expressed in dependency on the activation state of a macrophage involved in an inflammatory process, as exemplified in the Examples hereinbelow.

Briefly, such a differentially expressed nucleic acid sequence of the invention is identified by comparative expression profiling experiments using a cell or cellular extract from a hyperactivated macrophage, i.e. for example from the site of inflammation in COPD and from the corresponding site of control being not suffering from said disease, however, suffering under the same irritating condition like cigarette smoke exposure.

In a second step the proteins are identified which are encoded by the differentially expressed nucleic acid sequences, i.e. proteins playing a role in mediating the hyperactivation or in maintaining the hyperactivated state. A class of differentially expressed nucleic acid sequences of the invention can be identified to encode a class of proteins which act as nuclear receptor protein of the invention which is characterized in that it is expressed in a macrophage that is hyperactivated according the invention at a lower or higher level than the control level in a macrophage which is not hyperactivated. Such a protein of the invention is hereinafter named NHR-protein (“nuclear hormone receptor protein”).

A preferred example of a NHR-protein according to the present invention is estrogen-related receptor α (ERRα, SEQ ID NO:1 and 7) or nuclear receptor subfamily 4 group A member 1 (NR4A1, SEQ ID NO:2 and 8), depicted in the sequence listing.

The biological activity of a NHR-protein according to the present invention, i.e. mediating the involvement of a macrophage in an inflammatory process according to the invention, is dependent, for example, on recognition of and/or binding to a responsive DNA element influencing transcription of a DNA connected with said DNA element (e.g. ERRα responsive element (ERRE) or steroidogenic factor responsive element (SFRE)) or for example on recognition of and/or binding to an other protein resulting in a rendered transcription activity. The biological activity of a NHR-protein according to the invention is not limited to influence transcription activity. Besides this, the biological activity of a NHR-protein according to the invention for example facultatively comprises the regulation of apoptosis through a mechanism independent of transcription activity.

The invention also concerns functional equivalents, derivatives, variants, mutants and fragments of a NHR-protein, preferentially of the preferred proteins mentioned hereinbefore. Functional in this context means having a function of the respective corresponding NHR-protein which is involved in its biological activity, e.g. DNA and/or protein recognition.

According to the present invention, the biological activity of a NHR-protein expressed at a lower level than the control level is preferably activated in order to inhibit hyperactivation or reduce a hyperactivated state of a macrophage, whereby the biological activity of a NHR-protein which is expressed at a higher level than the control level is preferably inhibited in order to inhibit hyperactivation or reduce a hyperactivated state of a macrophage.

In one embodiment the present invention concerns a test method for determining whether a substance is an activator or inhibitor of a NHR-protein. Since a NHR-protein is involved in a chronic inflammatory airway disease and plays a role in mediating inflammation, a substance modulating the biological activity of a NHR-protein can be used for treating a chronic inflammatory airway diseases or can be used as lead compound for optimization of the function of the substance in a way that the optimized substance is suitable for treating chronic inflammatory airway diseases.

A method for determining whether a substance is an activator or an inhibitor of a function of a NHR-protein deregulated in a hyperactivated macrophage can be characterized in that the method comprises contacting the NHR-protein or variant, mutant or fragment thereof having a NHR-protein function with a substance to be tested whether it is an inhibitor or activator of a desired function of the NHR-protein, and measuring whether the desired function is inhibited or activated.

The desired function can be the biological activity of a NHR-protein of the invention.

Said measuring can be perfomed directly e.g. with well known procedures allowing to measure direct binding of a said protein with a said substance, or indirectly, e.g. using well known reporter systems allowing to draw conclusions about the binding of a said protein with a said substance.

For performing a method of the invention, a test system according to the invention can be used.

The present invention also concerns a test system for determining whether a substance is an activator or an inhibitor of a NHR-protein function. A test system useful for performing a method of the invention comprises a cellular or a cell-free system. For example, one embodiment of the invention concerns a test system that is designed in a way to allow the testing of substances acting on the expression level of the differentially expressed nucleic acid sequence e.g. using expression of a reporter-gene, e.g. luciferase gene or the like, as a measurable readout. Another embodiment of the invention concerns a test system that is designed in a way to allow the testing of substances directly interacting with a function, e.g. the recognition and/or binding activity, of the NHR-protein or interfering with the activation of a function of the NHR-protein by a natural or an artificial but appropriate activator of the NHR-protein, e.g. an appropriate ligand.

A test system of the invention comprises, for example, elements well known in the art. For example, cell-free systems may include, for example, a NHR-protein or a functional equivalent, derivative, variant, mutant or fragment of a NHR-protein, a nucleic acid encoding a NHR-protein or encoding a functional equivalent, derivative, variant, mutant or fragment of a NHR-protein in soluble or bound form or in cellular compartments or vesicles. Suitable cellular systems include, for example, a suitable prokaryotic cell or eukaryotic cell, e.g. such comprising a NHR-protein or a functional equivalent, derivative, variant, mutant or fragment of a NHR-protein, a nucleic acid encoding a NHR-protein or encoding a functional equivalent, derivative, variant, mutant or fragment of NHR-protein. A cell suitable for use in a said test system of the invention may be obtained by recombinant techniques, e.g. after transformation or transfection with a recombinant vector suitable for expression of a desired NHR-protein or functional equivalent, derivative, variant, mutant or fragment of a NHR-protein, or may e.g. be a cell line or a cell isolated from a natural source expressing a desired NHR-protein or functional equivalent, derivative, variant, mutant or fragment of NHR-protein. A test system of the invention may include a natural or artificial ligand of a NHR-protein if desirable or necessary for testing whether a substance of interest is an inhibitor or activator of a NHR-protein.

A test method according to the invention comprises measuring a read-out, e.g. a phenotypic change in the test system, for example, if a cellular system is used a phenotypic change of the cell is monitored. Such change may be a change in a naturally occurring or artificial response, e.g. a reporter gene expression of the cell to NHR-protein activation or inhibition, e.g. as detailed in the Examples hereinbelow.

A test method according to the invention can on the one hand be useful for high throughput testing suitable for determining whether a substance is an inhibitor or activator of the invention, but also e.g. for secondary testing or validation of a hit or lead substance identified in high throughput testing.

The present invention also concerns a substance identified in a method according to the invention to be an inhibitor or activator of a NHR-protein. A substance of the present invention is any compound which is capable of modulating preferably activating or inhibiting a function of a NHR-protein according to the invention. An example of a way to activate or inhibit a function of a NHR-protein is by influencing the expression level of said NHR-protein. Another example of a way to activate or inhibit a function of a NHR-protein is to apply a substance directly binding the NHR-protein and thereby activating or blocking functional domains of said NHR-protein, which can be done reversibly or irreversibly, depending on the nature of the substance applied.

Accordingly, a substance useful for activating or inhibiting biological activity of a NHR-protein includes a substance acting on the expression of differentially expressed nucleic acid sequence, for example a nucleic acid fragment hybridizing with the corresponding gene or regulatory sequence and thereby influencing gene expression.

Therefore, the invention concerns, for example, a substance which is a nucleic acid sequence coding for the gene of a NHR-protein, or a fragment, derivative, mutant or variant of such a nucleic acid sequence, which nucleic acid sequence or a fragment, derivative, mutant or variant thereof is capable of influencing the gene expression level, e.g. a nucleic acid molecule suitable as antisense nucleic acid, ribozyme, or for triple helix formation.

The invention also concerns a substance which is e.g. an antibody or an organic or inorganic compound directly binding to or interfering with the activation of a NHR-protein or directly binding to a NHR-protein and thereby affecting its biological activity.

In a further aspect, the present invention relates to a method for determining an expression level of a NHR-protein by determining the level of a nucleic acid coding for a NHR-protein, more preferably determining the level of respective messenger RNA, or determining the level of a NHR-protein itself, in a cell, preferably in a macrophage, more preferably in a macrophage isolated form a site of inflammation, even more preferably from a site of inflammation in a subject suffering from a chronic inflammatory airway disease. Such a method can be used, for example, for testing whether a substance is capable of influencing differentially expressed nucleic acid sequence expression levels in a method outlined above for determining whether a substance is an activator or inhibitor according to the present invention. A method for determining an expression level according to the invention can, however, also be used for testing the activation state of a macrophage, e.g. for diagnostic purposes or for investigation of the success of treatment for a disease which is caused by the hyperactivated macrophage, e.g. for monitoring. Said macrophage is preferably a mammalian, more preferably a human cell. Accordingly, macrophages of the present invention are preferably obtainable from the site of inflammation in a mammal and more preferably from a site of inflammation in a human being. Accordingly, the invention also relates to a method for diagnosis of a chronic inflammatory disease, or monitoring of such disease, e.g. monitoring success in treating beings in need of treatment for such disease, comprising determining an expression level of a nucleic acid coding for a NHR-protein, preferably messenger RNA, or a NHR-protein itself in a macrophage.

The present invention also relates to the use of a substance according to the invention for the treatment for a chronic inflammatory airway disease. Another embodiment of the present invention relates to a pharmaceutical composition comprising at least one of the substances according to the invention determined to be an activator or an inhibitor. The composition may be manufactured in a manner that is itself known, e.g. by means of conventional mixing, dissolving, granulating, dragee-making, levigating, powdering, emulsifying, encapsulating, entrapping or lyophilizing processes.

In order to use substances activating or inhibiting according to the invention as drugs for treatment for chronic inflammatory airway diseases, the substances can be tested in animal models for example an animal suffering from an inflammatory airway disorder or a transgenic animal expressing a NHR-protein according to the invention.

Toxicity and therapeutic efficacy of a substance according to the invention can be determined by standard pharmaceutical procedures, which include conducting cell culture and animal experiments to determine the IC₅₀, LD₅₀ and ED₅₀. The data obtained are used for estimating the animal or more preferred the human dose range, which will also depend on the dosage form (tablets, capsules, aerosol sprays ampules, etc.) and the administration route (for example transdermal, oral, buccal, nasal, enteral, parenteral, inhalative, intratracheal, or rectal).

A pharmaceutical composition containing at least one substance according to the invention as an active ingredient can be formulated in conventional manner. Methods for making such formulations can be found in manuals, e.g. “Remington Pharmaceutical Science”. Examples for ingredients that are useful for formulating at least one substance according to the present invention are also found in WO 99/18193, which is hereby incorporated by reference.

In a further aspect the invention concerns a method for treating a chronic inflammatory airway disease according to the invention. Such method comprises administering to a being, preferably to a human being, in need of such treatment a suitable amount of a pharmaceutical composition comprising at least one substance determined to be an activator or inhibitor by a method according to the invention for determining whether a substance is an activator or an inhibitor of a NHR-protein according to the invention.

In an other embodiment the invention relates to a method for selectively modulating NHR-protein concentration in a macrophage, comprising administering a substance determined to be an activator or inhibitor of a NHR-protein according to the invention.

The following examples are meant to illustrate the present invention, however, shall not be construed as limitation. However, the Examples describe most preferred embodiments of the invention.

### EXAMPLES

### Example 1

### Comparative Expression Profiling

The following is an illustration of how comparative expression profiling can be performed in order to identify a NHR-protein

Selection of Patients

Three groups of subjects are studied: healthy non-smokers, healthy smokers and patients with COPD.

In order to assess lung function subjects have to perform spirometry. A simple calculation based on age and height is used to characterize the results. COPD subjects are included if their FEV₁% predicted is <70%. Healthy smokers are age and smoking history matched with the COPD subjects but have normal lung function. Healthy non-smokers have normal lung function and have never smoked. The latter group has a methacholine challenge to exclude asthma. This technique requires increasing doses of methacholine to be given to the subject, with spirometry between each dose. When the FEV₁ falls 20% the test is stopped and the PC₂₀ is calculated. This is the dose of methacholine causing a 20% fall in FEV₁ and we will require a value of >32 as evidence of absence of asthma. All subjects have skin prick tests to common allergens and are required to have negative results. This excludes atopic individuals. The clinical history of the subjects is monitored and examined in order to exclude concomitant disease.

BAL (bronchoalveolar lavage) Procedure

Subjects are sedated with midazolam prior to the BAL. Local anaesthetic spray is used to anaesthetize the back of the throat. A 7 mm Olympus bronchoscope is used. The lavaged area is the right middle lobe. 250 ml of sterile saline is instilled and immediately aspirated. The resulting aspirate contains macrophages.

BAL Processing

BAL is filtered through sterile gauze to remove debris. The cells are washed twice in HBSS, resuspended in 1 ml HBSS (Hank's Balanced Salt Solution) and counted. The macrophages are spun to a pellet using 15 ml Falcon blue-cap polypropylen, resuspended in Trizol reagent (Gibco BRL Life Technologies) at a concentration of 1 ml Trizol reagent per 10 million cells and then frozen at −70° C.

Differential Gene Expression Analysis

Total RNA is extracted from macrophage samples obtained according to Example 1.3. Cell suspensions in Trizol are homogenized through pipetting and incubated at room temperature for 5 minutes. 200 μl chloroform per ml Trizol is added, the mixture carefully mixed for 15 seconds and incubated for 3 more minutes at room temperature. The samples are spun at 10000 g for 15 minutes at 4° C. The upper phase is transferred into a new reaction tube and the RNA is precipitated by adding 0.5 ml isopropanol per ml Trizol for 10 minutes at room temperature. Then, the precipitate is pelleted by using a microcentifuge for 10 minutes at 4° C. with 10000 g, the pellet is washed twice with 75% ethanol, air dried and resuspended in DEPC-H₂O.

An RNA cleanup with Qiagen RNeasy Total RNA isolation kit (Qiagen) is performed in order to improve the purity of the RNA. The purity of the RNA is determined by agarose gelelectrophoresis and the concentration is measured by UV absorption at 260 nm.

5 μg of each RNA is used for cDNA synthesis. First and second strand synthesis are performed with the SuperScript Choice system (Gibco BRL Life Technologies). In a total volume of 11 μl RNA and 1 μl of 100 μM T7-(dt)₂₄ primer (sequence: ggccagtgaa ttgtaatacg actcactata gggaggcggt ttttttttt tttttttttt ttt, SEQ ID NO:9) are heated up to 70° C. for 10 minutes and then cooled down on ice for 2 minutes. First strand buffer to a final concentration of 1×, DTT to a concentration of 10 mM and a dNTP mix to a final concentration of 0.5 mM are added to a total volume of 18 μl. The reaction mix is incubated at 42° C. for 2 minutes and 2 μl of Superscript II reverse transcriptase (200 U/μl) are added. For second strand synthesis 130 μl of a mix containing 1.15×second strand buffer, 230 μM dNTPs, 10 U E. coli DNA ligase (10 U/μl), E. coli DNA polymerase (10 U/μl), RNase H (2 U/μl) is added to the reaction of the first strand synthesis and carefully mixed with a pipette. Second strand synthesis is performed at 16° C. for 2 hours, then 2 μl of T4 DNA polymerase (5 U/μl) are added, incubated for 5 minutes at 16° C. and the reaction is stopped by adding 10 μl 0.5 M EDTA.

Prior to cRNA synthesis the double stranded cDNA is purified. The cDNA is mixed with an equal volume of phenol:chloroform:isoamylalcohol (25:24:1) and spun through the gel matrix of phase lock gels (Eppendorf) in a microcentrifuge in order to separate the cDNA from unbound nucleotides. The aqueous phase is precipitated with ammoniumacetate and ethanol. Subsequently, the cDNA is used for in vitro transcription. cRNA synthesis is performed with the ENZO BioArray High Yield RNA Transcript Labeling Kit according to manufacturer's protocol (ENZO Diagnostics). Briefly, the cDNA is incubated with 1×HY reaction buffer, 1×biotin labeled ribonucleotides, 1×DTT, 1×RNase Inhibitor Mix and 1×T7 RNA Polymerase in a total volume of 40 μl for 5 hours at 37° C. Then, the reaction mix is purified via RNeasy columns (Qiagen), the cRNA precipitated with ammonium acetate and ethanol and finally resuspended in DEPC-treated water. The concentration is determined via UV spectrometry at 260 nm. The remaining cRNA is incubated with 1×fragmentation buffer (5×fragmentation buffer: 200 mM Tris acetate, pH 8.1, 500 mM KOAc, 150 mM MgOAc) at 94° C. for 35 minutes.

For hybridization of the DNA chip 15 μg of cRNA is used, mixed with 50 pM biotin-labeled control B2 oligonucleotide, sequence: gtcgtcaaga tgctaccgtt cagga (SEQ ID NO: 10), 1×cRNA cocktail, 0.1 mg/ml herring sperm DNA, 0.5 mg/ml acetylated BSA, 1×MES (2-[N-morpholino]-ethanesulfonic acid) hybridization buffer in a total volume of 300 μl. The hybridization mixture is heated up to 99° C. for 5 minutes, cooled down to 45° C. for 10 minutes and 200 μl of the mix are used to fill the probe array. The hybridization is performed at 45° C. at 60 rpm for 16 hours.

After the hybridization the hybridization mix on the chip is replaced by 300 μl non-stringent wash buffer (100 mM MES, 100 mM NaCl, 0.01% Tween 20). The chip is inserted into an Affymetrix Fluidics station and washing and staining is performed according to the EukGE-WS2 protocol. The staining solution per chip consists of 600 μl 1×stain buffer (100 mM MES, 1 M NaCl, 0.05% Tween 20), 2 mg/ml BSA, 10 μg/ml SAPE (streptavidin phycoerythrin) (Dianova), the antibody solution consists of 1×stain buffer, 2 mg/ml BSA, 0.1 mg/ml goat IgG, 3 μg/ml biotinylated antibody.

After the washing and staining procedure the chips are scanned on the HP Gene Array Scanner (Hewlett Packard).

Data Analysis is performed by pairwise comparisons between chips hybridized with RNA isolated from COPD smokers and chips hybridized with RNA isolated from healthy smokers.

The following is an illustration of differentially expressed genes and their function as identified according to the approach of the present invention.

### Example 2

### ERRα (Estrogen-Related Receptor α)

A gene that is identified as consistently up-regulated in individuals with COPD codes for the estrogen-related receptor α (ERRα). Estrogen-related receptor alpha is an orphan member of the superfamily of nuclear hormone receptors. It binds to a single consensus half-site of the ERRα response element (ERRE) and to the steroidogenic factor 1 response element (SFRE) (Vanacker, J. -M., et al., EMBO J. 18:4270-4279 (1999)). It is found that genes with these sites in their promoter region are targets for transcription activation by ERRα. These genes are for example medium-chain acyl coenzyme A dehydrogenase (MCAG), osteopontin, and the thyroid hormone receptorα (Vanacker, J. -M., et al., Oncogene 17:2429-2435 (1998)). Due to the activation of MCAG it is assumed that ERRα is involved in regulating the energy balance in vivo (Sladek, R., et al., Mol. Cell. Biol. 17:5400-5409 (1997)).

The ERRα (acc. L38487) is consistently found upregulated (42%) in COPD smokers compared to healthy smokers. This is shown “fold change” values (Table 1). The p values for two separate groups comparing COPD smokers and healthy smokers are 0.03 and 0.15.

TABLE 1
Fold change values (FC) for comparisons between obstructed
smoker and healthy smokers. On average is upregulated by
2.3 fold, the median is 1.6 fold.

| Comp | FC | comp | FC | comp | FC | comp | FC |
|---|---|---|---|---|---|---|---|
| 1 vs 2 | −1.5 | 5 vs 43 | 4.8 | 39 vs 57 | 1.7 | 68 vs 66 | 3.4 |
| 1 vs 37 | 4.8 | 5 vs 56 | 1.0 | 39 vs 58 | 1.0 | 68 vs 69 | 2.4 |
| 1 vs 43 | 6.1 | 5 vs 57 | 2.4 | 39 vs 62 | 1.0 | 68 vs 76 | 2.9 |
| 1 vs 56 | 9.3 | 5 vs 58 | 1.0 | 44 vs 2 | −1.8 | 68 vs 78 | 3.5 |
| 1 vs 57 | 1.5 | 5 vs 62 | 1.0 | 44 vs 37 | 1.0 | 70 vs 65 | −1.5 |
| 1 vs 58 | 2.7 | 6 vs 2 | −2.1 | 44 vs 43 | 1.1 | 70 vs 66 | 1.5 |
| 1 vs 62 | 5.0 | 6 vs 37 | 3.2 | 44 vs 56 | 1.1 | 70 vs 69 | 1.1 |
| 3 vs 2 | −1.9 | 6 vs 43 | 3.5 | 44 vs 57 | −1.5 | 70 vs 76 | 1.3 |
| 3 vs 37 | 4.0 | 6 vs 56 | 5.6 | 44 vs 58 | 1.0 | 70 vs 78 | 1.6 |
| 3 vs 43 | 4.8 | 6 vs 57 | 1.0 | 44 vs 62 | 1.0 | 71 vs 65 | 1.3 |
| 3 vs 56 | 7.7 | 6 vs 58 | 1.9 | 64 vs 65 | −1.2 | 71 vs 66 | 6.1 |
| 3 vs 57 | 1.2 | 6 vs 62 | 3.4 | 64 vs 66 | 1.9 | 71 vs 69 | 6.1 |
| 3 vs 58 | 2.2 | 39 vs 2 | −1.3 | 64 vs 69 | 1.3 | 71 vs 76 | 6.0 |
| 3 vs 62 | 4.2 | 39 vs 37 | 1.0 | 64 vs 76 | 1.6 | 71 vs 78 | 6.7 |
| 5 vs 2 | 1.1 | 39 vs 43 | 6.1 | 64 vs 78 | 2.0 | | |
| 5 vs 37 | 1.0 | 39 vs 56 | 1.0 | 68 vs 65 | 1.5 | | |

Cloning of ERRα

ERRα is cloned from a total RNA extracted from human kidney. 5 μg RNA is reverse transcribed into cDNA with 5 ng oligo(dt)₁₈ primer, 1×first strand buffer, 10 mM DTT, 0.5 mM dNTPs and 2 U Superscript II (Gibco BRL) at 42° C. for 50 minutes. Then, the reaction is terminated at 70° C. for 15 minutes and the cDNA concentration is determined by UV-spectrophotometry. For amplification of ERRα 100 ng of the cDNA and 10 pmoles of sequence-specific primers for ERRα (forward primer: ggggacaagt ttgtacaaaa aagcaggcta tgggattgga gatgagctc; SEQ ID NO:3 and reverse primer: ggggaccact ttgtacaaga aagctgggtt cagtccatca tggcctcgag SEQ ID NO:4) are used for PCR. Reaction conditions are: 2 minutes of 94° C., 35 cycles with 30 seconds at 94° C., 30 seconds at 53° C., 90 seconds at 72° C., followed by 7 minutes at 72° C. with Taq DNA-polymerase. The reaction mix is separated on a 2% agarose gel, a band of about 1000 bp is cut out and purified with the QIAEX II extraction kit (Qiagen). The concentration of the purified band is determined and about 120 ng are incubated with 300 ng of pDONR201, the donor vector of the Gateway system (Life Technologies), 1×BP clonase reaction buffer, BP clonase enzyme mix in a total volume of 20 μl for 60 minutes at 25° C. Then, reactions are incubated with 2 μl of proteinase K and incubated for 10 minutes at 37° C. The reaction mix is then electroporated into competent DB3.1 cells and plated on Kanamycin-containing plates. Clones are verified by sequencing. A clone, designated pDONR-ERRα, with identical sequence to the database entry (acc. X51416) is used for further experiments.

Generation of a Transfection Vector for ERRα

The vector containing ERRα described under 1.1. is used to transfer the cDNA for ERRα to the expression vector pcDNA3.1 (+)/attR that contains the “attR1” and “attR2” recombination sites of the Gateway cloning system (Life Technologies) where ERRα is expressed under the control of the CMV promoter. 150 ng of the “entry vector” pDONR-ERRα is mixed with 150 ng of the “destination vector” pcDNA3.1(+)/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells 450 μL of S.O.C. is added and cells are incubated at 37° C. for 60 minutes. Cells (100 μl) are plated on LB plates containing 100 μg/ml ampicillin and incubated over night.

A colony that contains pcDNA3.1(+)/attR with ERRα as an insert is designated pcDNA/ERRα and used for transfection studies.

Transfection of ERRα

Monocytic cell lines are seeded in a 35 mm petri dish and cultivated in RPMI 1640 media containing 10% FCS supplemented with 100 U/ml penicillin, 100 μg/ml streptomycin, 2 mM glutamine, and 1×non-essential amino acids over night. Cells that are grown to a confluency of 50-80% are used for transfection. 6 μl FuGene6 (Roche Biochemicals) is added to 100 μl of culture media without serum and equilibrated for 5 minutes at room temperature. Then, 2 μg of purified pcDNA/ERRα is added to the prediluted FuGene6 solution, gently mixed, and further incubated at room temperature for 15 minutes. The media is aspirated from the cells and 4 ml of fresh media is added to the cells. The FuGene6/DNA solution is added dropwise to the cells and distributed evenly by swirling of the media. After 48 hours the media is aspirated and replaced by RPMI 1640, 10% FCS, 2 mM glutamine, 100 U/ml penicillin, 100 μg/ml streptomycin, and 200 μg/ml G418. During the following five days the media is replaced daily until dead cells and debris is washed out until single colonies of cells are visible. Single colonies are isolated by separation with cloning cylinders and releasing them from the surface by addition of 100 μl of 1×trypsin/EDTA. Cells are transferred from the cloning cylinders to 4 ml of media and plated in 6 well-plates. Single clones are expanded and the expression of ERRα in several clones is tested via Western blotting. A cell clone with the highest expression of ERRα is used for further studies.

Expression of Recombinant ERRα

The vector containing ERRα described under 1.1. is used to transfer the cDNA for ERRα to the expression vectors gpET28abc/attR that contains the “attR1” and “attR2” recombination sites of the Gateway cloning system (Life Technologies). These vectors allow the expression of recombinant his-tagged ERRα in bacteria under the control of the T7 promoter. 150 ng of the “entry vector” pDONR-ERRα is mixed with 150 ng of the “destination vector” gpET28abc/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells 450 μl of S.O.C. is added and cells are incubated at 37° C. for 60 minutes. Cells (100 μl) are plated on LB plates containing 100 μg/ml ampicillin and incubated over night.

A colony that contains gpET28abc/attR with ERRα fused to the his-tag in the correct reading frame is designated gpET/ERRα and used for expression of ERRα in bacteria.

Purification of Recombinant ERRα

1 l LB broth including 100 μg/ml ampicillin is inoculated with 0.5 ml of an overnight culture of E. coli M15(pREP4) that carries pDONR-ERRα. The culture is incubated at 37° C. with vigorous shaking until OD₆₀₀ of 0.6. Expression is induced by adding 1 mM IPTG and the culture is grown further for 4 hours. Cells are harvested by centrifugation at 4000×g for 20 minutes at 4° C. Pellet is frozen at −20° C.

Cells are thawed on ice and resuspended in 2 ml/g cell pellet of lysis buffer (50 mM NaH₂PO4, pH 8.0, 300 mM NaCl, 10 mM imidazole). Then, lysozyme is added to 1 mg/ml and incubated on ice for 30 minutes. Then, cells are sonicated (six bursts of 10 seconds at 300 W). 10 μg/ml RNase A and 5 μg/ml DNase I is added and incubated on ice for 10 minutes. Then, lysates are cleared by spinning debris at 10000×g for 20 minutes at 4° C. Then, protease inhibitors (40 μg/ml bacitracin, 4 μg/ml leupeptin, 4 μg/ml chymostatin, 10 μg/ml pefabloc, 100 μM PMSF) are added. 3 ml of Ni-NTA resin (Qiagen) are added to the lysate and filled into a column. Binding to the resin is allowed for 60 minutes at 4° C. during gentle shaking. Then, column outlet is opened, the resin washed twice with 12 ml wash buffer (50 mM NaH₂PO4, pH 8.0, 300 mM NaCl, 20 mM imidazole) and eluted with four times 3 ml of elution buffer (50 mM NaH₂PO4, pH 8.0, 300 mM NaCl, 250 mM imidazole). The elution fraction that contains the recombinant protein is determined by SDS-PAGE and protein concentration of the purified protein is determined by the method of Bradford.

Fluorescence Polarisation Assay (Especially for ERRα)

The fluorescence polarisation assay is used in order to find substances that directly inhibit the interaction of the nuclear hormone receptor with its DNA binding site. Two complementary oligos containing the binding site (AGGTCA) for ERRα are synthesized. One oligo: acgggtagag gtcactgtga cctctacccg (SEQ ID NO:5) is synthesized with TAMRA-labeled thymidin. The complementary oligo: cgggtagagg tcacagtgac ctctacccgt (SEQ ID No.6) is synthesized without label.

In order to anneal both oligos, 10 μM of the TAMRA-labeled oligo and 15 μM of the complementary oligo are mixed in 10 mM Tris/HCl, pH 7.5, 80 mM NaCl, 1 mM EDTA. Oligos are incubated at 95° C. for 5 minutes (reaction tube in a 2 1 beaker filled with boiling water) and cooled down to room temperature over night. DNA-binding assays are performed in 96-well Fluotrac 200 plates (Greiner). Per well 150 μl 20 nM of the annealed oligo are incubated with 40 nM of the nuclear hormone receptor in a reaction buffer containing 10 mM Tris/HCl, pH 7.5, 100 mM NaCl, 0.1 mM EDTA, 1% glycerol. Binding is allowed at 27° C. for 2 hours. Substances according to the invention are added in a concentration range from 0.1-100 ng/ml. Fluorescence is monitored with a Polarion fluorometer (Tecan). Wells including binding buffer and oligo are used as controls. 1 nM fluorescein is used to calibrate the fluorometer.

Phenotypic/Cellular Effects Caused by ERRα

The following assays are performed with cell lines, e.g. THP-1 (Tsuchiya, S., et al., Int. J. Cancer 26:171-176 (1980)), MonoMac 6 (Ziegler-Heitbrock, H. W., et al., Int. J. Cancer 41:456-461 (1988)) that are transiently or stably transfected with ERRα and the read-outs are compared to mock-transfected cells. Additionally, substances according to the invention are added in order to inhibit the effects caused by ERRα.

Production and Release of Cytokines

Monocytic/macrophage cell lines are stimulated with various stimuli, like 10 nM PMA, 20 ng/ml M-CSF, 20 ng/ml GM-CSF, 20 μg/ml LPS (from Salmonella minnessota Re595) at cell densities between 2.5 and 5×10⁵ cells/ml. Cells are harvested after 0, 1, 3, 6, 12, 24, 48, and 72 hours, the supernatant frozen for further investigation, cells are washed with PBS, and resuspended in 400 μl of RLT buffer (from Qiagen RNeasy Total RNA Isolation Kit) with 143 mM β-mercaptoethanol, the DNA sheared with a 20 g needle for at least 5 times and stored at −70° C.

Stimulation of cells by cigarette smoke is performed by a smoke-enriched media. 100 ml RPMI media without supplements is perfused with the cigarette smoke of 2 cigarettes. The smoke of the cigarettes is pulled into a 50 ml syringe (about 20 volumes of a 50-ml volumes per cigarette) and then perfused into the media. Afterwards, the pH of the media is adjusted to 7.4, and the media is filtersterilized through a 0.2 μm filter. Cells are resuspended in smoke-enriched media and incubated for 10 minutes at 37° C. at a density of 1×10⁶ cells/ml. Then, cells are washed twice with RPMI 1640 and seeded in flasks or 24-well plates (MonoMac 6) for the times indicated above.

Total RNAs are isolated with the Qiagen RNeasy Total RNA Isolation Kit (Qiagen) according to the manufacturer's protocol. Purified RNA is used for TaqMan analysis. The expression levels of cytokines TNFα, IL-1β, IL-8, and IL-6 are measured.

Detection of Secreted Cytokines

Proteins in the supernatants of the cultured and stimulated cells are precipitated by adding TCA to a final concentration of 10%. Precipitates are washed twice with 80% ethanol and pellets are resuspended in 50 mM Tris/HCl, pH 7.4, 10 mM MgCl₂, 1 mM EDTA. Protein concentration is determined via the Bradford method and 50 μg of each sample are loaded on 12% SDS polyacrylamide gels. Gels are blotted onto PVDF-membranes, blocked for 1 hour in 5% BSA in TBST, and incubated for 1 hour with commercially available antibodies against human TNFα, IL-1β, IL-8, and IL-6. After washing with TBST blots are incubated with anti-human IgG conjugated to horseradish-peroxidase, washed again and developed with ECL chemiluminescence kit (Amersham). Intensity of the bands are visualised with BioMax X-ray films (Kodak) and quantified by densitometry.

Detection of Secreted Matrix Metalloproteases and Other Proteases

The procedure is identical to the one used for cytokines. Antibodies used for Western blotting are against human MMP-1, MMP-7, MMP-9, and MMP-12.

Activity of Secreted Matrix Metalloproteases

Protease activity is determined with a fluorescent substrate. Supernatants isolated from stimulated and unstimulated cells (described above) are incubated in a total volume of 50 μl with 1 μM of the substrate Dabcyl-Gaba-Pro-Gln-Gly-Leu-Glu(EDANS)-Ala-Lys-NH2 (Novabiochem) for 5 minutes at room temperature. Positive controls are performed with 125 ng purified MMP-12 per reaction. Protease activity is determined by fluorometry with an excitation at 320 nm and an emission at 405 nm.

In an alternative assay to determine proteolytic activity and cell migration a chemotaxis (Boyden) chamber is used. In the wells of the upper part of the chamber cells (10⁵ cells per well) are plated on filters coated with an 8 μm layer of Matrigel (Becton Dickinson). In the lower compartment chemoattractants like leukotriene B₄ (10 ng/ml), MCP-1(10 ng/ml) are added to the media. After five days filters are removed, cells on the undersurface that have traversed the Matrigel are fixed with methanol, stained with the Diff-Quik staining kit (Dade Behring) and counted in three high power fields (400×) by light microscopy.

Chemotaxis Assay

In order to determine chemotaxis a 48 well chemotaxis (Boyden) chamber (Neuroprobe) is used. Cells are starved for 24 hours in RPMI media without FCS. Chemoattractants, (50 ng/ml IL-8 , 10 ng/ml MCP-1, 10 nM lipoxin A4, leukotriene B₄ (10 ng/ml), MCP-1 (10 ng/ml) and substances according to the invention are diluted in RPMI media without FCS and 30 μl is placed in the wells of the lower compartment. The upper compartment is separated from the lower compartment by a polycarbonate filter (pore size 8 μm). 50 μl cell suspension (5×10⁴) are placed in the well of the upper compartment. The chamber is incubated for 5 hours at 37° C. in a humidified atmosphere with 5% CO₂. Then the filter is removed, cells on the upper side are scraped off, cells on the downside are fixed for 5 minutes in methanol and stained with the Diff-Quik staining set (Dade Behring). Migrated cells are counted in three high-power fields (400×) by light microscopy.

Adherence Assay

Cells are harvested, washed in PBS and resuspended (4×10⁶/ml) in PBS and 1 μM BCECF ((2′-7′-bis-(carboxethyl)-5(6′)-carboxyfluorescein acetoxymethyl) ester (Calbiochem), and incubated for 20 minutes at 37° C. Cells are washed in PBS and resuspended (3.3×10⁶/ml) in PBS containing 0.1% BSA. 3×10⁵ cells (90 μl) are added to each well of a 96-well flat bottom plate coated with laminin (Becton Dickinson) and allowed to settle for 10 minutes. Substances according to the invention are added and plates are incubated for 20 minutes at 37° C. Cells are washed with PBS containing 0.1% BSA and adherent cells are solubilized with 100 μl of 0.025 M NaOH and 0.1% SDS. Quantification is performed by fluorescence measurement.

Phagocytosis

Cell suspensions (2.5×10⁴ cells/ml) are seeded in 6-well plates with 5 ml of U937 or THP-1 or in 24-well plates with 2 ml of MonoMac6 and incubated for 1 hour at 37° C. in a humidified atmosphere with 5% CO₂ in the presence of substances according to the invention. 40 μl of a dispersed suspension of heat-inactivated Saccharomyces boulardii (20 yeast/cell) are added to each well. Cells are incubated for three more hours, washed twice with PBS and cytocentrifuged. The cytospin preparations are stained with May-Grünwald-Giemsa and phagocytosed particles are counted by light microsopy.

Determination of Energy Balance (Especially for ERRα)

Acidification of the medium by cells due to metabolic processes are monitored via the Cytosensor microphysiometer system (Molecular Devices GmbH, Gräfelfing, Germany). 10⁶ cells of a monocytic cell line stably expressing VP16/ERRα, a constitutive active chimera of ERRα (Sladek et al. 1997), are transiently transfected with NRRE-MCAD-LUC (Sladek et al. 1997), a medium-chain acyl coenzyme A dehydrogenase-luciferase reportergene construct containing NRRE1 (nuclear receptor response element 1) as a binding site for ERRα and grown for 24 hours. Then cells are seeded in a capsule of the cytosensor in RPMI 1640, 2.5% FCS and grown over night at 37° C. in 5% CO₂ in a humidified atmosphere. Before use, cells are washed with serum-free RPMI 1640, 10 mM HEPES (pH 7.4). Substances according to the invention (0.1-100 ng/ml) are added at time zero. Inhibition of MCAD-mediated and ERRα-driven acidification of the medium is monitored over a period of 120 minutes with cells treated with serum-free RPMI 1640, 10 mM HEPES (pH 7.4) set as 100%.

### Example 3

### NR4A1 (nuclear receptor subfamily 4, group A, member 1)

A gene that is identified as consistently downregulated in individuals with COPD codes for NR4A1 which is an orphan member of the nuclear hormone receptor superfamily of transcription factors. It mediates cell proliferation in response to growth factors in the nucleus. Besides, NR4A1 also regulates apoptosis through a mechanism independent of transcriptional activity. In response to apoptotic stimuli, NR4A1 is translocated from the nucleus to the cytoplasm, where it targets mitochondria to induce cytochrome release and apoptosis (Li, H., et al., Science 289:1159-1164 (2000)).

NR4A1 (acc. D49728) is consistently found downregulated (44%) in COPD smokers compared to healthy smokers. This is shown by “fold change” values (Table 2). The p values for comparing two groups of COPD smokers and healthy smokers are 0.15 and 0.009.

TABLE 2
Fold change values (FC) for comparisons between obstructed
smoker and healthy smokers. On average NR4A1 is downregulated
by 1.6 fold, the median is −1.4 fold.

| Comp | FC | comp | FC | comp | FC | comp | FC |
|---|---|---|---|---|---|---|---|
| 1 vs 2 | 1.0 | 5 vs 43 | −3.1 | 39 vs 57 | 1.0 | 68 vs 66 | 1.0 |
| 1 vs 37 | 1.0 | 5 vs 56 | −2.7 | 39 vs 58 | −2.4 | 68 vs 69 | −6.8 |
| 1 vs 43 | −1.2 | 5 vs 57 | 1.0 | 39 vs 62 | −1.3 | 68 vs 76 | −3.4 |
| 1 vs 56 | 1.0 | 5 vs 58 | −4.6 | 44 vs 2 | 1.7 | 68 vs 78 | −5.7 |
| 1 vs 57 | 1.0 | 5 vs 62 | −3.1 | 44 vs 37 | −1.2 | 70 vs 65 | −5.0 |
| 1 vs 58 | −2.1 | 6 vs 2 | 1.0 | 44 vs 43 | −1.1 | 70 vs 66 | 1.0 |
| 1 vs 62 | 1.0 | 6 vs 37 | 1.0 | 44 vs 56 | 1.1 | 70 vs 69 | −6.1 |
| 3 vs 2 | 1.0 | 6 vs 43 | −4.5 | 44 vs 57 | 3.1 | 70 vs 76 | −3.2 |
| 3 vs 37 | 1.0 | 6 vs 56 | −5.4 | 44 vs 58 | −2.0 | 70 vs 78 | −5.2 |
| 3 vs 43 | −2.4 | 6 vs 57 | 1.0 | 44 vs 62 | 1.0 | 71 vs 65 | −5.0 |
| 3 vs 56 | −2.0 | 6 vs 58 | −9.0 | 64 vs 65 | −1.5 | 71 vs 66 | 1.0 |
| 3 vs 57 | 1.0 | 6 vs 62 | −7.5 | 64 vs 66 | 1.0 | 71 vs 69 | −5.9 |
| 3 vs 58 | −4.3 | 39 vs 2 | 1.0 | 64 vs 69 | −1.7 | 71 vs 76 | −3.0 |
| 3 vs 62 | −2.2 | 39 vs 37 | 1.0 | 64 vs 76 | 1.1 | 71 vs 78 | −4.9 |
| 5 vs 2 | 1.0 | 39 vs 43 | −1.4 | 64 vs 78 | −1.5 | | |
| 5 vs 37 | 1.0 | 39 vs 56 | −1.1 | 68 vs 65 | −5.7 | | |

The protein is cloned and assays are performed in an analogous manner to the cloning and assays described hereinbefore.

Apoptosis Assay (Especially for NR4A1)

The assay to determine the number of apoptotic cells is performed with the terminal transferase kit by Roche Diagnostics (cat. No. 220582). Cell lines stably expressing the nuclear hormone receptor are seeded in 8-well tissue culture plates with 5×10⁴ per ml and stimulated with PMA (100 ng/ml) to induce apoptosis. Simultaneously, substances according to the invention are added to the cells ranging from 1 to 1000 ng/ml. 3 to 6 hours after stimulation cells are washed with PBS, 1 mM MgCl₂, fixed with 3% paraformaldehyde in PBS for 10 minutes and treated twice with PBS/50 mM NH₄Cl for 5 minutes. Then, cells are treated with for 5 minutes with PBS/0.5% Triton X-100 at room temperature. Then, cells are washed twice with PBS and equilibrated with 1×transferasebuffer for 10 seconds. After removing the transferasebuffer, 20 μl of reaction mix (200 μl reaction mix consists of 40 μl 5×reaction buffer, 20 μl 25 mM CaCl₂, 1 μl dNTP rhodamin (tetramethylrhodamin-5-2‘-desoxy-uridin-’6′-triphosphate), 2 μl (2U) terminal transferase, 137 μl H₂O bidest.) are added, covered with parafilm, and incubated for 1 hour at room temperature without access of light in a humidified atmosphere. The reaction is stopped by adding 50 mM EDTA, 50 mM EGTA, rinsed for 2 minutes in PBS and incubated for 5 minutes at room temperature. The samples are air dried and covered by glycerol. The percentage of labeled cells that represent apoptotic cells is determined by fluorescence microscopy.

All documents, e.g., scientific publications, patents and patent publications, recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method for determining whether a substance is an activator or an inhibitor of a function of a NHR-protein deregulated in a hyperactivated macrophage, characterized in that the method comprises contacting the NHR-protein or variant, mutant or fragment thereof having a NHR-protein function with a substance to be tested whether it is an inhibitor or activator of a desired function of the NHR-protein, and measuring whether the desired function is inhibited or activated.

2. A method according to claim 1 in which the inhibition or activation of the desired function is measured directly.

3. A method according to claim 1 in which the inhibition or activation of the desired function is measured indirectly.

4. A method according to claim 1 in which the NHR-protein is a mammalian NHR-protein.

5. A method according to claim 4 in which the NHR-protein is a human NHR-protein.

6. A method according to claim 1 in which the analysis is performed using a cellular system.

7. A method according to claim 1 in which the analysis is performed using a cell-free system.

8. A method according to claim 1 in which said NHR-protein is selected from the group consisting of ERRα (SEQ ID NO:1); and NR4 μl (SEQ ID NO:2).

9. A method according to claim 8 in which ERRα (SEQ ID NO:1) is used or a variant, mutant or fragment thereof having the same function.

10. A method according to claim 8 in which NR4A1 (SEQ ID NO:2) is used or a variant, mutant or fragment thereof having the same function.

11. A method according to claim 1 in which the function is DNA recognition and or DNA binding.

12. A method according to claim 1 in which the function is protein recognition and or protein binding.

13. A method for determining an expression level of a NHR-protein deregulated in a hyperactivated macrophage comprising determining the expression level of NHR-protein expressed in a macrophage.

14. A method according to claim 13 in which said macrophage is a mammalian macrophage.

15. A method according to claim 14 in which said macrophage is a human macrophage.

16. A method according to claim 13 in which said NHR-protein is selected from the group consisting of ERRα (SEQ ID NO:1); and NR4A1 (SEQ ID NO:2).

17. A method according to claim 13 or 16 in which said expression level is determined by determining the level of nucleic acid coding for a NHR-protein in a macrophage.

18. A method according to claim 13 or 16 in which said expression level is determined by determining the level of a NHR-protein.

19. A method according to anyone of claims 13-18 for diagnosis or monitoring of a chronic inflammatory airway disease.

20. A method according to claim 19 in which the chronic inflammatory airway disease is selected from the group consisting of chronic bronchitis and COPD.

21. A method according to claim 13 in which the analysis is performed using a macrophage or a part thereof obtainable from the site of inflammation.

22. A test system for determining whether a substance is an activator or an inhibitor of a function a NHR-protein deregulated in a hyperactivated macrophage comprising al least a NHR-protein or a variant, or a mutant, or a fragment thereof having a NHR-protein function.

23. A test system according to claim 22 in which said NHR-protein is selected from the group consisting of ERRα (SEQ ID NO:1); and NR4A1 (SEQ ID NO:2).

24. A test system according to claim 23 comprising a cell expressing a NHR-protein.

25. A substance determined to be an activator or inhibitor of a NHR-protein deregulated in a hyperactivated macrophage.

26. A substance which is an activator or inhibitor of a NHR-protein deregulated in a hyperactivated macrophage for the treatment for a disease.

27. A substance according to claim 26 in which said disease is a chronic inflammatory airway disease.

28. A substance according to claim 27 in which said chronic inflammatory airway disease is selected from the group consisting of chronic bronchitis and COPD.

29. A pharmaceutical composition comprising at least one substance determined to be an activator or inhibitor of a NHR-protein deregulated in a hyperactivated macrophage.

30. Use of a substance determined to be an activator or inhibitor of a NHR-protein for preparing a pharmaceutical composition for treating a chronic inflammatory airway disease.

31. Use of a substance according to claim 30 in which the chronic inflammatory airway disease is selected from the group consisting of chronic bronchitis and COPD.

32. A method for treating a chronic inflammatory airway disease which method comprises administering to a being in need of such treatment a suitable amount of a pharmaceutical composition comprising at least one substance determined to be an activator or inhibitor of a NHR-protein.

33. A method according to claim 32 for treating a mammal.

34. A method according to claim 32 for treating a human being.

35. A method according to claim 32 for treating a chronic inflammatory airway disease selected from the group consisting of chronic bronchitis and COPD.

36. A method for selectively modulating a NHR-protein in a macrophage, comprising administering a substance determined to be an activator or inhibitor of a NHR-protein.

37. A method according to claim 36 in which the macrophage is involved in a chronic inflammatory airway disease.

38. A method according to claim 37 in which the chronic inflammatory airway disease is selected from the group consisting of chronic bronchitis and COPD.
